(19) United States
(12) Patent Application Publication
(10) Pub. No.: US 2022/0143432 A1
(43) Pub. Date: May 12, 2022

(54) PROTECTIVE RESPIRATORY MASK

(71) Applicants: Jaime C. SALLES, Croton-on-Hudson, NY (US); Fernando J. SALLES, Scotch Plains, NJ (US)

(72) Inventors: Jaime C. SALLES, Croton-on-Hudson, NY (US); Fernando J. SALLES, Scotch Plains, NJ (US)

(21) Appl. No.: 17/430,389

(22) PCT Filed: Feb. 14, 2020
(86) PCT No.: PCT/US20/18324

§ 371 Date: Aug. 12, 2021

Related U.S. Application Data
(60) Provisional application No. 62/805,371, filed on Feb. 14, 2019.

(51) Int. Cl. A62B 18/02; A62B 9/06

(52) U.S. Cl. CPC A62B 18/025; A62B 9/06

(57) ABSTRACT

A breathing apparatus configured to be worn on the face of a person is disclosed. The apparatus comprises a breathing manifold having an internal space with openings for a connection to the nasal passage and mouth of the user and openings for one or more gas-exchange ports. An armature holds the manifold in position relative to the face and provides pressure against the nose and mouth to provide a sealed interface between the internal volume of the apparatus and each of the nose and mouth. According to a salient aspect, the armature is configured to securely hold the breathing apparatus to the person's face without requiring additional securements such as head straps. The armature also includes one or more attachment points for supporting other components of the breathing apparatus including, for example, an air-inlet filter and an exhaust valve, through which air can be exchanged.

### CROSS-REFERENCE TO RELATED APPLICATION

The present application claims the benefit of U.S. Provisional Application No. 62/805,371, to Salles et al., titled Novel Protective Respiratory Mask, filed on Feb. 14, 2019 with the U.S. Patent and Trademark Office, which is hereby incorporated by reference in its entirety.

### FIELD OF THE DISCLOSURE

The present invention relates to breathing apparatuses and, in particular, protective breathing masks configured to be received in the user's mouth.

### BACKGROUND

Respirators and protective breathing masks (from herein out referred to simply as “masks” but taken to mean the same as respirators, surgical masks, safety breathing devices, etc.) are a critical type of personal protection equipment that needs to conform to certain standards to ensure that they function as intended for the safety of the user. As an example, in the US a surgical mask can be rated and certified by the National Institute of Occupational Safety and Health (NIOSH), according to the mask's chemical resistance and filtering capacity (e.g. an “N95” certification designates that the mask filters greater than 95% of non-oily particles that are 0.3 microns or greater including infectious agents; where as an N100 would filter 100% of particles 0.3 microns or greater). Although a mask is certified, it still requires adjustment by the individual wearer and generally requires fit testing at the time of usage to ensure and appropriate seal on the face of the individual. Fit testing is important to ensure a close proper fit and to minimize the amount of leakage of the mask at the seals on the face as this would compromise the mask's certified rating. E.g. face masks do not seal properly when an individual has facial hair—therefore the recommendation is to remove all facial hair if you need to use the mask. Additionally, to ensure a proper fit and filtration, most if not all existing masks are rightfully designed to ensure safety to their certified rating at the expense of all other variables including communication and comfort. For example, most surgical masks and respirators require cranial or ear straps to hold the mask in place; these straps tend to slip over time requiring readjustment and become uncomfortable on the ears or head. Additionally, masks capture humidity and heat from expired air which impinges on the skin surface of the face and adds to discomfort especially over long term wear.

In short, there are multiple challenges associated with current mask and respirator designs:

1. The seal on the face: Masks are minimally effective in preventing infections by a healthy wearer for a variety of reasons including incomplete sealing on the face. Human face topography is quite variable limiting a more universal fit especially in instances with facial hair. Additionally, the seal on the face can feel tenuous even when still, more so when grimacing, speaking or moving. The mask's fit and seal can also be easily disturbed by simple actions, e.g. speaking, rubbing and scratching. These challenges require that masks are fit tested to ensure the mask is properly sealing and functioning—improving the quality ease and strength of the seal and obviating or reducing the need for fit testing would be beneficial.

2. Sizing and fit: Masks have to come in various sizes to cover the variations in face and age. Separate masks are designed for children. Additionally, there is no standardization in fit between manufacturers of masks. Taking advantage of the reduced variation in mouth to nose features enables one to cover a larger range of the population with one sized mask.

3. Not re-usable: Most surgical-type masks are currently envisioned as single-use disposable devices. Developing a secure reusable safety mask with quick interchangeable filters would be beneficial.

4. Communication: Certain masks impinge on vocal communication. Additionally, masks generally diminish the conveyance of emotion and empathy from caregiver or health care provider given its large footprint on the face. Enabling verbal and non-verbal communication would be beneficial in multiple settings including the healthcare setting where empathy and human interaction is critical to wellbeing.

5. Comfort: Although generally sacrificed to safety, masks tend to be uncomfortable over even short periods due to heat and moisture on the face and pressure on bridge of the nose and on ears/head caused by straps, glues and other adherence mechanisms. Improving short-term and long-term comfort while strictly maintaining or improving safety would improve adherence.

It is with respect to these and other considerations that the disclosure made herein is presented.

### SUMMARY OF THE DISCLOSURE

According to an aspect of the present invention, a breathing apparatus is disclosed. The breathing apparatus comprises a manifold. The manifold includes a body defining an internal chamber and a sealing mouthpiece extending from the body. The sealing mouthpiece has an upper wall configured to be received in the anterior upper oral vestibule (the space between the lips/cheeks and the gums/teeth) of the mouth and a lower wall configured to be received in the lower anterior lower oral vestibule of the mouth. The upper and lower walls are configured to sealingly engage an internal surface of the mouth. The mouthpiece also includes a central passageway located between the upper and lower walls. The central passageway is generally in register with an opening of the mouth, and provides fluidic communication between the oral cavity and the internal chamber of the manifold. Furthermore, the body comprises one or more ports through which gasses can be exchanged from the internal chamber.

The breathing apparatus further comprises a nasal base extending from the body. The nasal base is configured to bear against the person's nose. Additionally, the nasal base is shaped to define one or more hollow passages providing fluidic communication between the person's nares and nasal passages and the internal chamber of the manifold during use.

The breathing apparatus further comprises a lower clip configured to be placed over the person's lower lip, the lower lip clip. The lower lip clip has a first end arranged for placement within the lower anterior oral vestibule and a second end arranged to bear against an external surface of the person's face, namely, the chin. The lower lip clip distributes a load of the apparatus against the chin and the lower lip clip is sized and shaped so as to limit interference with the lower lip muscles during use.

The breathing apparatus further comprises an upper clip configured to be placed over the person's upper lip, the upper lip clip. The upper lip clip has a first end arranged for placement within the upper anterior oral vestibule. The upper lip clip also has a second end, situated external to the person's mouth, that is mounted to the nasal base. Additionally, the upper lip clip is sized and shaped to locate the nasal base against the underside of the person's nose and to limit interference with the upper lip muscles during use.

According to a further aspect, a device for holding a breathing apparatus to a mouth of a person is disclosed. The device comprises a nasal base configured to bear against an underside of a nose of a person and an upper lip clip configured to be placed over an upper lip of the person. In particular, the upper lip clip includes a first end arranged for placement within an upper anterior oral vestibule, and a second end provided external to the mouth during use. The nasal base is mounted to the second end, and the upper lip clip is sized and shaped to locate the nasal base against the underside of the person's nose and limit interference with the person's upper lip muscles during use.

The device further comprises a lower lip clip configured to be placed over the person's lower lip. In particular, the lower lip clip includes a first end arranged for placement within the lower anterior oral vestibule and a second end arranged to bear against an external surface of the person's chin and concentrate forces against the chin. The lower lip clip is sized and shaped to limit interference with the lower lip muscles during use.

Furthermore, the device comprises an expansion bow extending between the lower lip clip and one or more of the upper lip clip and the nasal base. In particular, the expansion bow is configured to exert an opposing force between the lower lip clip and one or more of the upper lip clip and the nasal base. Additionally, the device comprises one or more mounts for mounting the breathing apparatus to one or more of the expansion bow, the lower lip clip, the upper lip clip and the nasal base.

These and other aspects, features, and advantages can be appreciated from the accompanying description of certain embodiments of the invention and the accompanying drawing figures and claims.

### BRIEF DESCRIPTION OF THE DRAWING FIGURES

FIG. 1A includes front and side-view diagrams illustrating an exemplary breathing apparatus being worn on the face of a person in accordance with one or more embodiments.

FIG. 1B is an exploded diagram of the apparatus of FIG. 1A, shown from a front, left side perspective view.

FIG. 1C is a front view illustration of a person's face particularly the mouth and nose region and including anatomical references.

FIG. 1D is a cut-away side view of the apparatus of FIG. 1A while worn by a user, wherein the cross-section cut-line is labeled 1D in FIG. 1A.

FIG. 1E is a simplified side-view of the apparatus of FIG. 1A showing the internal volume of the manifold providing fluidic communication with the person's nasal passage and mouth cavity.

FIG. 1F is a cut-away side view of an isolated armature assembly of the apparatus of FIG. 1A shown being worn by a user.

FIG. 2A depicts a front-left side perspective view of a manifold in accordance with one or more embodiments.

FIG. 2B depicts a back-view of the manifold of FIG. 2A.

FIG. 2C shows the top view of the manifold of FIG. 2A.

FIG. 2D depicts a bottom view of the manifold of FIG. 2A.

FIG. 2E shows a cut-away view of the manifold from the front view, wherein the manifold is cut along the line 2E shown in FIG. 2C and wherein the mouthpiece is shown in the neutral/resting position.

FIG. 2F shows a cut-away view of the manifold from the front view, wherein the manifold is cut along the line 2E shown in FIG. 2C and wherein the mouthpiece is shown in the closed configuration.

FIG. 2G shows a cut-away view of the manifold from the front view, wherein the manifold is cut along the line 2E shown in FIG. 2C and wherein the mouthpiece is shown in the fully open configuration.

FIG. 3A depicts a front-left side perspective view of a lower lip clip in accordance with one or more embodiments.

FIG. 3B depicts a back-view of the lower lip clip of FIG. 3A.

FIG. 4A depicts a front-left side perspective view of an upper lip clip in accordance with one or more embodiments.

FIG. 4B depicts a side-view of the upper lip clip of FIG. 4A.

FIG. 4C depicts a perspective view of the upper lip clip of FIG. 4A shown from the top-back view.

FIG. 5A depicts a front-left side perspective view of the nasal base and the upper lip clip in accordance with one or more embodiments.

FIG. 5B depicts a front view of the nasal base of FIG. 5A.

FIG. 5C depicts a back view of the nasal base of FIG. 5A.

FIG. 5D depicts the nasal base of FIG. 5A from the front-top perspective.

FIG. 5E depicts an exploded view of the nasal base of FIG. 5A and the upper lip clip of FIG. 4A shown from the back, left side perspective.

FIG. 6A depicts a front-left side perspective view of the expansion bow in accordance with one or more embodiments, wherein the expansion bow is show in both a resting state (left) and in a compression or loaded state (right).

FIG. 6B depicts an assembled armature comprising the expansion bow of FIG. 6A, in a loaded state, and the nasal base of FIG. 5A, the upper lip clip of FIG. 4A, and the lower lip clip of FIG. 3A, shown from a front left side perspective.

FIG. 6C depicts the assembled armature of FIG. 6B, shown from a side view.

FIG. 6D depicts the assembled armature of FIG. 6B, shown from a top left rear perspective view.

FIG. 7A is a front-left-side perspective view of the nasal shroud in accordance with one or more embodiments.

FIG. 7B is a cut-away side view of a nasal shroud of FIG. 7A.

FIG. 7C is a perspective view of a shroud of FIG. 7A.

FIG. 8 is an exploded diagram of an exemplary exhaust valve shown from a front, left side perspective view.

FIG. 9A is a rear-view of a filter in accordance with one or more embodiments.

FIG. 9B is a front left-side perspective view of a filter of FIG. 9A.

### DETAILED DESCRIPTION OF CERTAIN EMBODIMENTS OF THE DISCLOSURE

By way of overview and introduction, a breathing apparatus is disclosed. The apparatus is configured to be worn on the face of a user and, thus, can be referred to as a “mask.” The apparatus comprises a breathing manifold having an internal space with openings for a connection to the nasal passage and the mouth of the wearer. The manifold also includes openings for one or more gas-exchange ports. The manifold is supported by an armature. The armature functions to both hold the manifold in position relative to the face and provides pressure against the nose and mouth to provide a sealed interface between the manifold and each of the nose and mouth that prevents ambient air from entering/escaping through the seals to a suitable degree and to enable the individual wearing the mask to speak. The armature also includes one or more attachment points for supporting other components of the breathing apparatus including, for example, an air-inlet filter and an exhaust valve, through which air can be exchanged.

The armature can comprise an assembly of one or more components. According to a salient aspect, the armature is configured to securely hold the breathing apparatus to the person's face without requiring additional securements such as head/ear straps or adhesives. It should thus be understood that the armature can be a component of a breathing apparatus that includes, for example, a breathing manifold and air-delivery device as described herein. In addition, or alternatively, the armature can be considered a stand-alone device for holding any number of other breathing apparatuses to a person's face.

According to a salient aspect, the manifold and armature provide a respiratory safety device that provides benefits over existing designs through, inter alia, improved safety of the wearer by generating a better seal on the face; the elimination of facial hair as a barrier to safety in face masks; the elimination of head straps or other head restraining features; the maintenance of verbal and non-verbal facial communications; a reusable interface that can fit a larger percentage of the population without size variations; the reduction of heat issues and discomfort on long term use. While exemplary embodiments described herein are used as personal air filtration systems, it should be understood that the application is not so limited. The disclosed invention concepts can be applied to multiple types of respiratory safety masks including surgical masks, chemical safety masks, masks for protection from epidemics and the like. The disclosed invention concepts can also be applied to other gas-delivery systems, both positive and negative pressure systems.

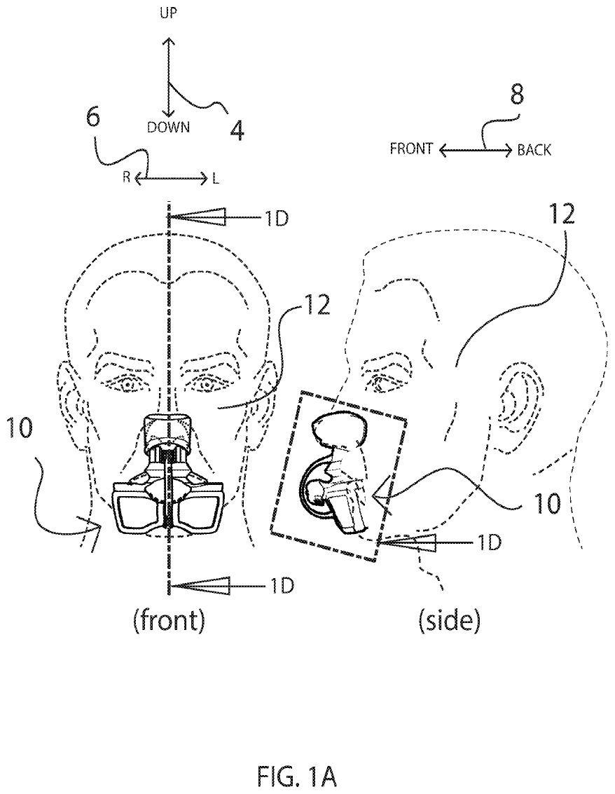

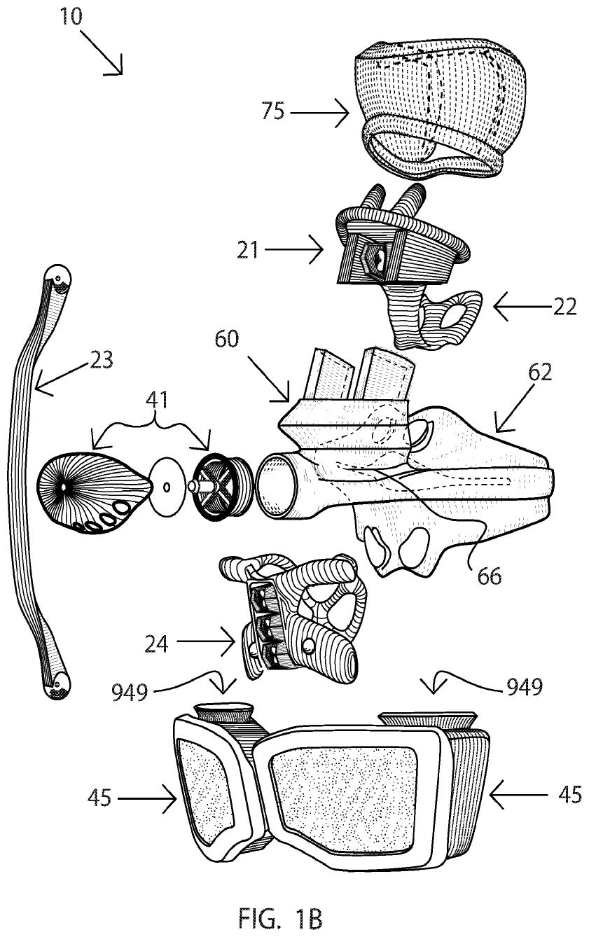

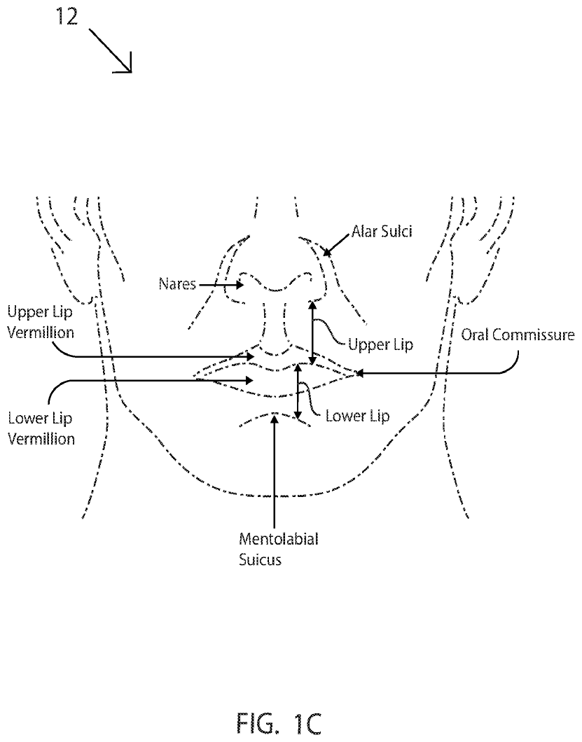

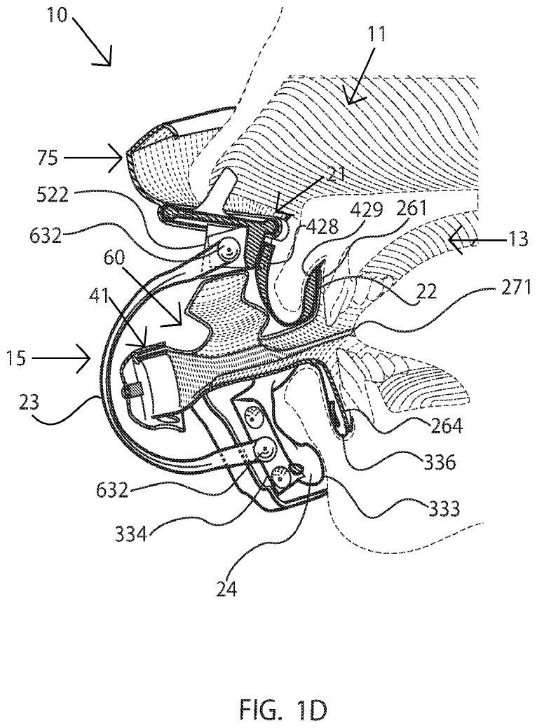

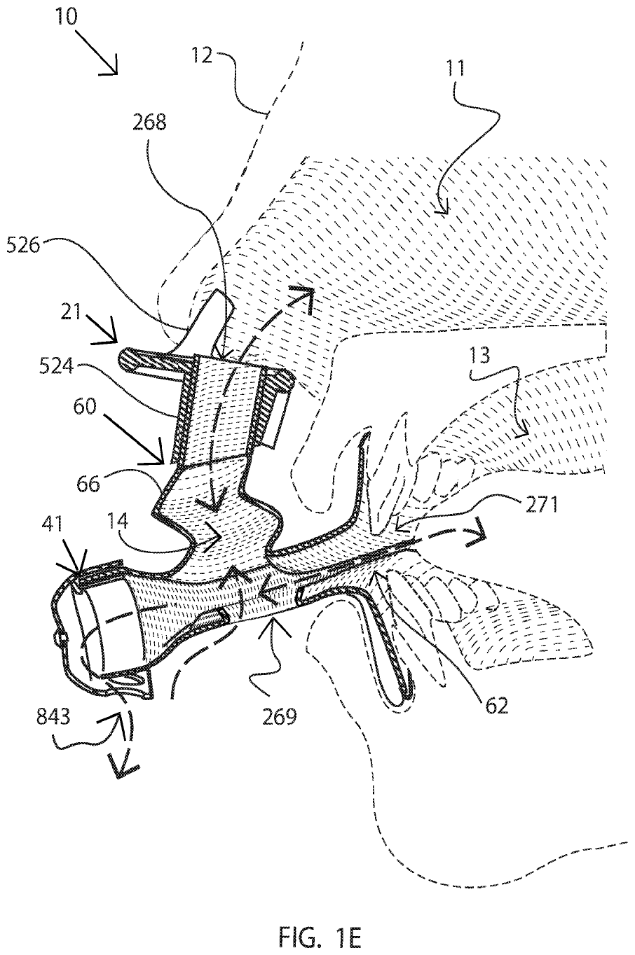

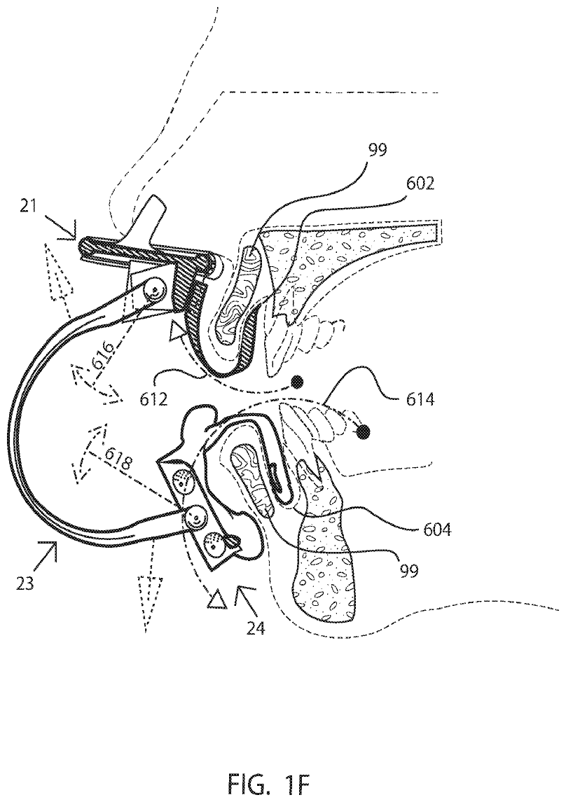

For clarity, certain terms used to reference the anatomy of the human body, particularly the lips, mouth and oral region are briefly defined below. FIG. 1C is a drawing illustrating a front-view of a face and annotated with some anatomical references.

Lips: The structures that surround the oral aperture. In the central region their superior border corresponds to the inferior margin of the base of the nose. Laterally, their limits follow the alar-nasal sulci and the upper and lower lips join at the oral commissures. The inferior limit of the lips in the central region is the mentolabial sulcus.

Vermilion: The red part of the lips.

Mouth: The oral aperture that opens into the oral cavity proper. The opening is bounded by the upper and lower vermilion. The cavity comprises the alveolar arches with gums and teeth, the hard and soft palate, and the tongue, anchored to the floor of the mouth. The oral cavity leads into the oropharynx, bounded by the tonsillar pillars.

Oral Commissure: The place, on each side of the lips, where the lateral aspects of the vermilion of the upper and lower lips join.

Oral Vestibule: sometimes referred to as labial fissure—Slit-like space between the lips/cheeks and the gums/teeth.

Oral Cavity: The space bounded superiorly by the hard and soft palates, laterally by the alveolar processes of the maxillary and mandibular bones, and inferiorly by the tongue.

Alveolar Ridge: The U-shaped bony crests of the upper and lower jaw in which the teeth are situated.

Gingiva (gums): Dense fibrous tissue covered by mucous membrane overlying the alveolar ridge in which the teeth are situated.

Labial Frenulum: A thin fold of soft tissue extending from the gingiva of the mid-anterior alveolar ridge to the inner surface of the medial part of the upper (upper labial frenulum) or lower (lower labial frenulum) lip.

Teeth: Hard dental structures located on the alveolar ridges and situated in the gingiva.

Nasal passage: Nasal passage refers to the air passageways through the nostrils of the nose.

Nares (Naris, singular)—the external portion of the nostrils.

Upper Anterior Oral Vestibule: the oral vestibule of the upper lip specifically relating to that portion behind the mouth.

Lower anterior oral vestibule: the oral vestibule of the lower lip specifically relating to that portion behind the mouth.

Lip muscles: the complex of muscles known as the orbicularis oris.

FIG. 1A includes front and side-view diagrams illustrating an exemplary breathing apparatus 10 being worn on the face 12 of a person in accordance with one or more embodiments. For clarity, FIG. 1A shows the side of the apparatus 10 referred to as the left side, when viewed from the perspective of the wearer, and shows the left side of the person's face. FIG. 1B is an exploded diagram of the apparatus 10 shown from a front, left side perspective view. FIG. 1D is a cut-away side view of the apparatus of FIG. 1A while worn by a user, wherein the cross-section cut-line is labeled 1D in FIG. 1A.

As shown in FIG. 1B, the breathing apparatus 10 comprises an upper lip clip 22, a nasal base 21, an expansion bow 23, a lower lip clip 24, a one way valve 41, two filters 45, a breathing manifold 60, and a nasal shroud 75. At least the nasal base 21 and the upper lip clip 22 define a structure referred to as the upper lip module (or assembly) 20. The armature 15 comprises at least the upper lip module 20, the lower lip clip 24 and the expansion bow 23. The upper lip assembly 20 can also include the nasal shroud 75. FIG. 1E is a simplified side-view of the apparatus of FIG. 1A showing the internal volume of the manifold providing fluidic communication with the person's nasal passage and mouth cavity. FIG. 1F is a cut-away side view of an isolated armature assembly of the apparatus of FIG. 1A shown being worn by a user.

Turning briefly to FIG. 1E, which is a cut-away, side-view diagram showing the apparatus 10 worn on the face 12 and illustrating the internal volume of the breathing apparatus 10 relative to the nasal passage 11 and mouth cavity 13 of the person. As shown, the breathing apparatus 10 has an internal space that is predominantly defined by the manifold 60, although air passageways can extend from the manifold to the nasal passage through the nasal base 21. The manifold 60 has a body 66 that defines the main air chamber/internal volume 14 connecting the person's oral cavity 13 and nasal passage 11. The manifold also includes a mouthpiece 62 configured to be received into the person's mouth, the mouthpiece is shaped to define a breathing tube having a central opening 271 providing fluidic communication between the oral cavity 13 and the internal volume 14 of the manifold. Extending radially outward from the central opening are walls of the mouthpiece configured to sealingly engage with an internal surface of the oral vestibule thereby forming an air-seal around the mouth.

As further described herein, the body 66 of the manifold 60 includes one or more gas-exchange ports through which gasses can be inhaled and/or expired. For instance, one or more one-way valves 41 can be connected to port(s) in the manifold such that gasses can be exhaled/expired out of the chamber 14. Similarly, one or more air-scrubbing filters 45 (not shown here but can be seen in FIG. 1B for example) can be connected to respective inlet ports 269 in the manifold such that ambient air can be inhaled into the chamber through the filters. While the exemplary configuration of apparatus 10 includes two filters 45 mounted to the manifold, it should be understood that alternative air filtration or gas supply systems can be connected to the manifold, for example, compressed air tanks. The flow of gasses into and out of the manifold, via its exterior openings/ports, and between the internal manifold volume 14 and each of the nasal passage 11 and the mouth cavity 13 are illustrated in FIG. 1E with large dashed arrows.

The nasal base 21 is configured to be pressed against the underside of the nose. It assists in providing the sealed fluid connection between the manifold and the nares and helps to maintain this connection during use. The nasal base 21 is shaped to sealingly mate with the upper air passage(s) of the manifold 60 (268 from the manifold 60 fitting into 524 on the nasal base 21). It is also shaped to include one or more passage(s) in registry with the nares. Accordingly, the fluidic communication between the nasal passage and the internal volume 14 of the manifold is provided through the nasal base. The nasal base also includes physical locating features that maintain the nasal base in registry with the person's nostrils during use. For instance, the nasal base includes two elongate protrusions 526 that are arranged to extend from its upper surface generally upward and into the nostrils so as to resist movement of the nasal base relative to the underside of the nose.

Because of the contour of the underside of a nose can vary from person to person, the sealed engagement between the device 10 and the nasal passage can be enhanced through incorporation of a nasal shroud 75. In the exemplary arrangement, the nasal shroud 75 extends upward from the periphery of the nasal base 21 and is shaped to encompass at least a portion of the nose. The nasal shroud 75 also can include compressible material that bears against the skin of the nose and forms an air-seal against the nose. By way of further example, the seal against the nares can be provided using other sealing structures mounted to the nasal base and held against the nares, for example, nasal pillows which are known in the art of CPAP masks.

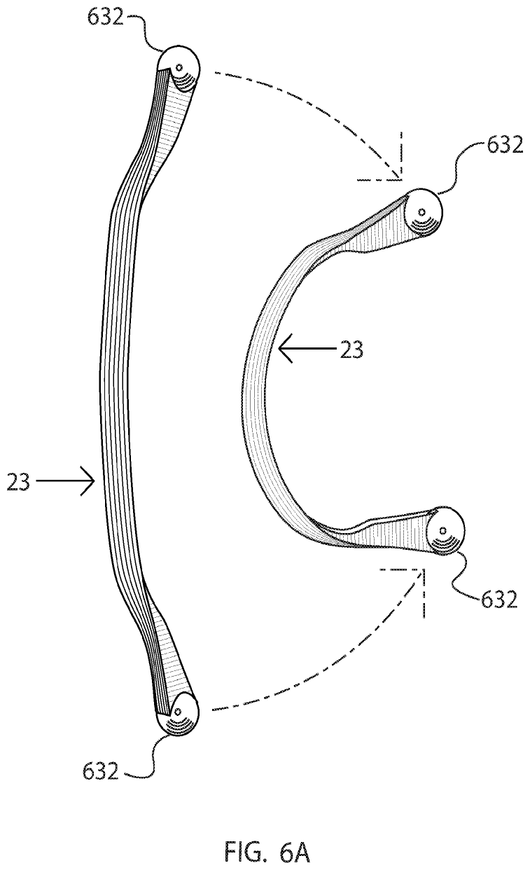

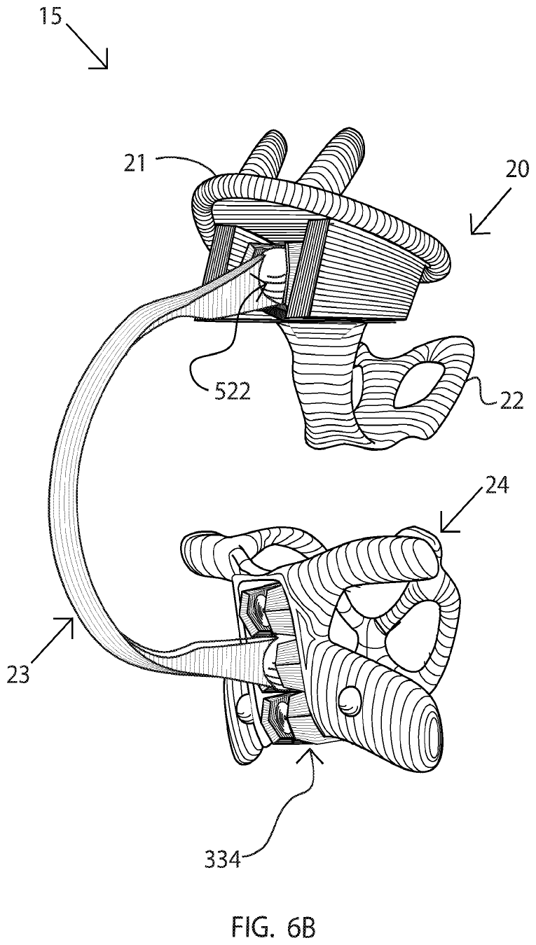

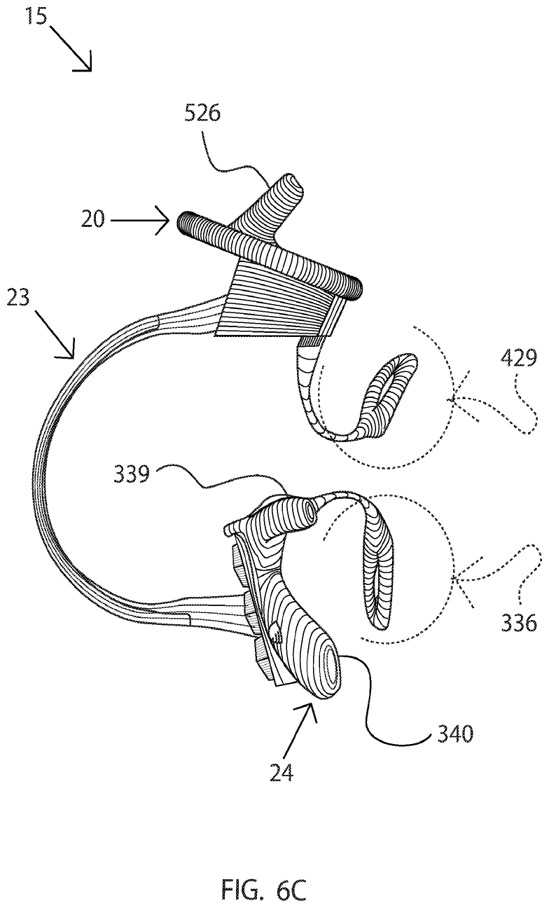

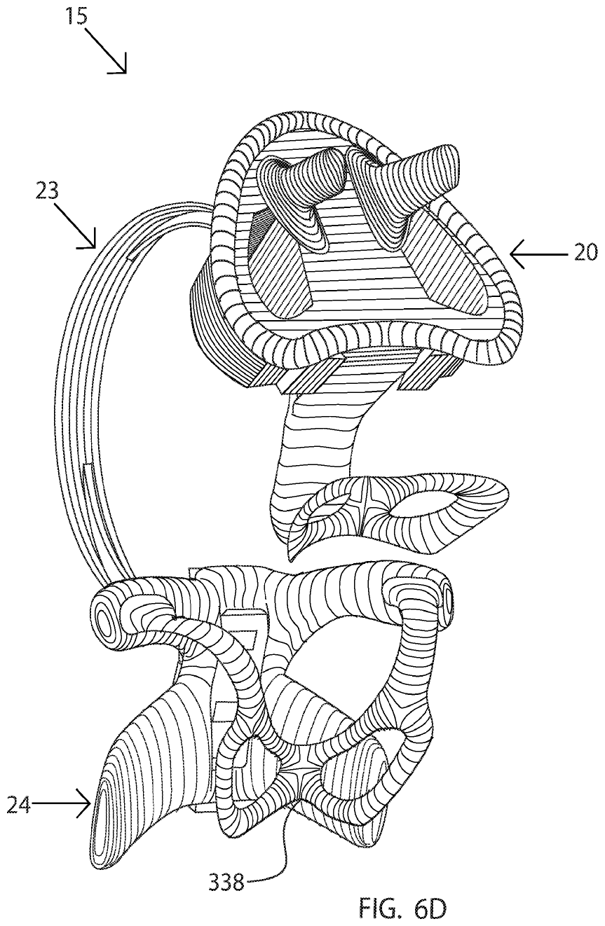

As noted, the manifold 60 can be supported by an armature 15 (shown in isolation in FIG. 6B) which can include a nasal upper lip module (assembly 20 in FIG. 6B comprising the nasal base 21 and upper lip clip 22), the lower lip clip 24 and the expansion bow 23. The armature is configured to hold the manifold in position relative to the face. It also exerts forces against the nose and mouth to provide a sealed interface between the apparatus 10 and each of the nose and mouth that prevents ambient air from entering/escaping through those interfaces to a suitable degree.

The upper lip clip 22 is a C-shaped structure configured to be fit around the upper lip. The upper lip clip is preferably worn at a central region (in the lateral direction 6) of the upper lip. When in place, it extends from one end located between the upper lip and gums or teeth (the upper oral vestibule) to a second end provided a location near the exterior surface of the upper lip toward the base of the nose. The upper lip clip 22 is placed around the upper lip near the open end and performs the function of locating the nasal base against the underside of the nose and, preferably, urging the back-end of the nasal base toward the intersection of the nose and the upper lip. The upper lip clip also can be mounted to a mouthpiece of the manifold and assist in providing a suitable air-seal between the mouthpiece and the inner surface of the mouth. The upper lip clip is configured to concentrate forces of the apparatus 10 toward the base of the nose and preferably at a location away from the lip muscles so as to limit interference with free movement of the lip muscles.

Similarly, the lower lip clip 24 is a C-shaped structure configured to be fit over the lower lip. The lower lip clip is preferably worn at a central region in the lateral direction 6 of the lower lip. When in place it extends from one end located between the lower lip and gums (lower oral vestibule) to a second end that preferably rests against the chin in the region at or near the mentolabial sulcus. The lower lip clip is configured to concentrate the weight and other forces from the apparatus 10 against the chin while limiting interference with free movement of the lip muscles. The upper lip clip can also be joined to the mouthpiece of the manifold to assist in providing a suitable air-seal between the mouthpiece and the inner surface of the mouth.

The expansion bow 23 is configured to connect the upper and lower portions of the armature. For example, it extends between the nasal base 21 and the lower lip clip 24. The expansion bow configured to exert an opposing, expansion force between these upper and lower portions of armature generally in the up and down direction 4. This expansion force urges the nasal base into its preferred position against the underside of the nose and urges the lower lip clip into position with the outer end of the lower lip clip against the chin.

It should be understood that while one or more of the components of apparatus 10 are described herein as being discrete structures that are combined and/or joined to define an assembly, any number of the components can be combined into a single structure. For example, the nasal base can be integrally formed with the upper clip. By way of further example, the lower clip can be integrally formed with the manifold. Similarly, in some configurations, the upper clip can be integrally formed with the manifold. Furthermore, such integrally formed structures can be formed of the same material, or can comprise a plurality of different materials that are joined.

Exemplary configurations of each of the aforementioned components of breathing apparatus 10 are further described herein in greater detail with continued reference to FIGS. 1A-1F.

### Breathing Manifold

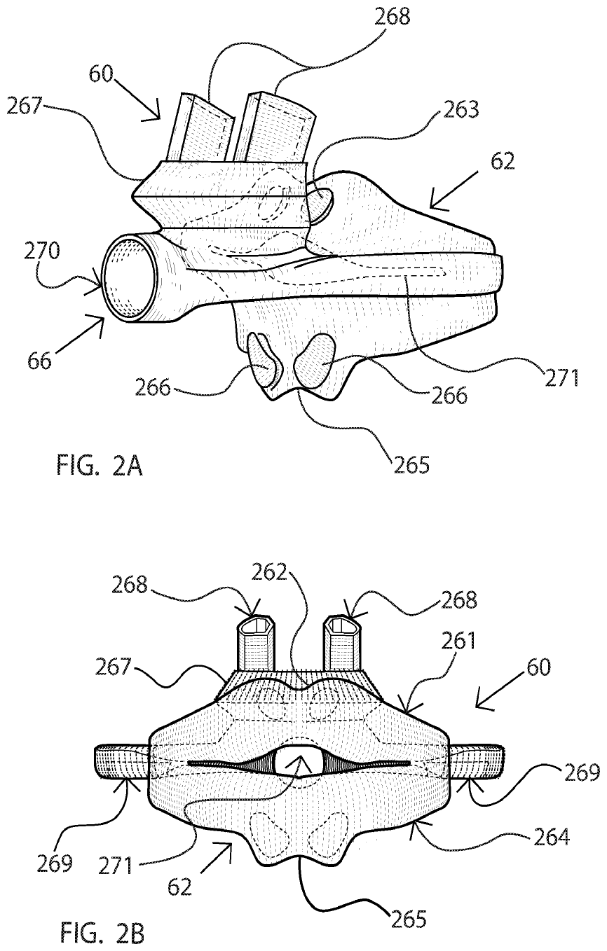

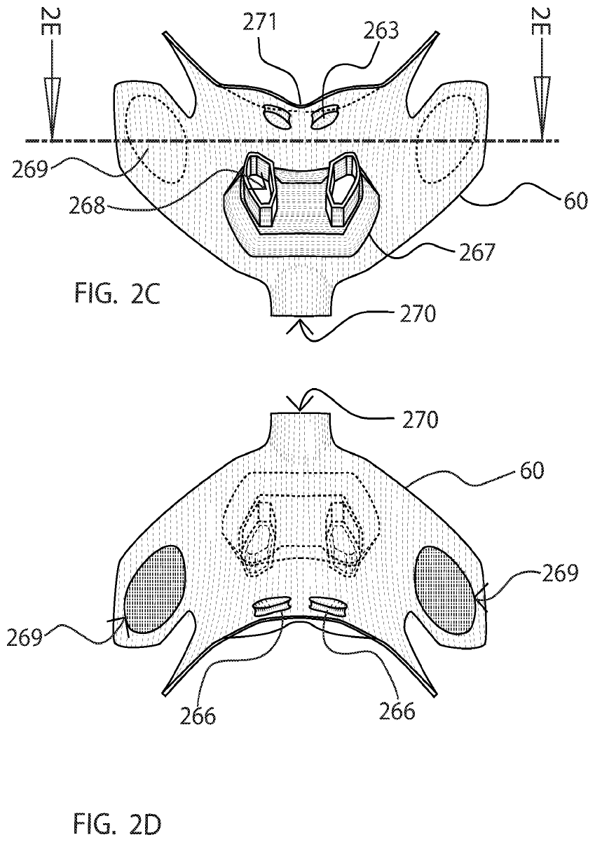

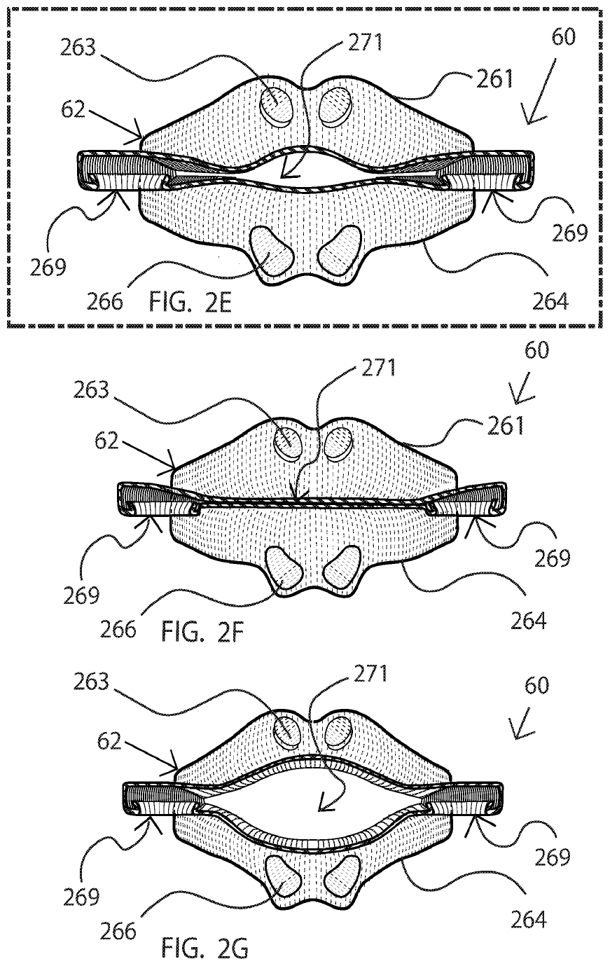

An exemplary configuration of the breathing manifold 60 in accordance with one or more embodiments is shown and described herein in connection with FIGS. 2A-2G.

FIG. 2A depicts a front-left side perspective view of the manifold. FIG. 2B depicts a back-view. FIG. 2C shows a top view. FIG. 2D depicts a bottom view. FIG. 2E shows a cut-away view of the manifold from the front view, wherein the manifold is cut along the line 2E shown in FIG. 2C showing the mouthpiece in the resting position. FIG. 2F shows a cut-away view of the manifold from the front view, wherein the manifold is cut along the line 2E shown in FIG. 2C, showing the mouthpiece in the closed position. FIG. 2G shows a cut-away view of the manifold from the front view, wherein the manifold is cut along the line 2E shown in FIG. 2C, showing the mouthpiece in open position.

In an exemplary arrangement, the breathing manifold 60 comprises a body 66 and a mouthpiece portion 62. The body of the manifold, which is the portion supported in front of the user's face during use, includes walls that bound a hollow internal volume or “cavity” 14. The mouthpiece 62, which extends from the body is configured to form a seal with the user's mouth in the anterior oral vestibule and defines an air passageway 271 between the mouth and the internal volume of the manifold. As such, the mouthpiece includes a distal portion that is shaped to be received into the user's mouth. This portion includes walls that extend outwardly from a central region and that are configured to sealingly engage with the internal surface of the user's mouth to create an air seal that extends entirely about the anterior portion of the oral vestibule. The seal can be formed by the walls bearing against one or more of the gums (gingiva) and the inner surface of the upper and lower lips and the internal surface of the mouth in the region where the upper and lower lips join (the oral commissures).

The mouthpiece 62 also includes a “breathing tube” in that the walls of the mouthpiece define a hollow central passageway 271 (as shown in, e.g., FIG. 2B). When worn by a user, the breathing tube extends from the manifold body 66 through the labial opening of the mouth. The central passageway thus provides fluidic communication between the mouth cavity and the internal volume 14 of the manifold thereby allowing gasses to be inspired or expired through the mouthpiece. Although described as a tube, the breathing tube is not required to have a round or tube-like shape. For example, in the arrangement shown and described in the figures, the mouthpiece has an opening 217 that is generally similar to the shape of the mouth orifice.

More specifically, the mouthpiece is, sized, shaped and made of a material that allows the shape and size of the opening 271 to be manipulated by the user's lips so that it mimics the varying shape of the mouth opening, which varies as the user is speaking, breathing and the like. For example, as shown in FIG. 2B, the mouthpiece 62 walls are shaped such that, as it extends from the manifold body 66 toward the free end inside the mouth, the walls generally follow the contour of a mouth thereby providing a central opening 271 shaped like the mouth orifice. As noted, the mouthpiece is preferably made of a flexible material. This allows the opening to be opened and closed and have a variable size and shape controlled by movement of the user's lip muscles and jaw. This configuration of the mouthpiece facilitates clarity of speech.

A variety of stable, non-toxic, medical grade, flexible and/or elastic materials, e.g. silicon, nitriles, vinyls, urethanes, etc., with varying densities throughout the construction of the manifold and mouthpiece can be used thereby enabling increased flexibility or increased rigidity/support where needed along the “breathing tube.”

As shown in FIG. 2A, elements of the mouthpiece, particularly the portion arranged to be received inside the user's mouth include an upper anterior oral vestibule seal 261. The seal 261 is a thin wall of the mouthpiece configured to be received inside the upper anterior oral vestibule of the mouth and bear against inner surface of the lips, cheeks and gums. The seal 261 also includes a superior labial frenulum notch 262, upper lip clip anchor studs 263. Similarly, the lower anterior oral vestibule seal 264 is a wall arranged to be received in the lower anterior oral vestibule of the mouth and includes an inferior labial frenulum notch 265 and lower lip clip anchor studs 266.

Upper anterior oral vestibule seal 261 (or “upper wall”) refers to the portion of the manifold that creates the air seal in the upper half of the mouth residing in the upper anterior oral vestibule. The upper anterior oral vestibule seal 261 is provided by a thin wall that extends radially from a central opening of the mouthpiece generally in an upward direction. The upper oral vestibule seal 261 is shaped to include a notch for the superior labial frenulum 262. It also includes an attachment means allowing it to be physically joined to the upper lip clip which, in this exemplary case includes two studs 263 extending from a surface of the wall and which can be pressure fit into complementary shaped upper lip anchor cut outs 430, further described herein. Specifically, the superior labial frenulum notch 262 is the notch formed in the distal end of the sealing wall 261 to allow room for the user's superior labial frenulum. The upper lip clip anchor studs 263 are the mechanical mounting mechanism configured to physically attach the upper wall of the mouthpiece to the upper lip clip 22. In this configuration, the anchor studs comprise two flexible form fitting studs that pressure fit into the upper lip anchor cut outs (430).

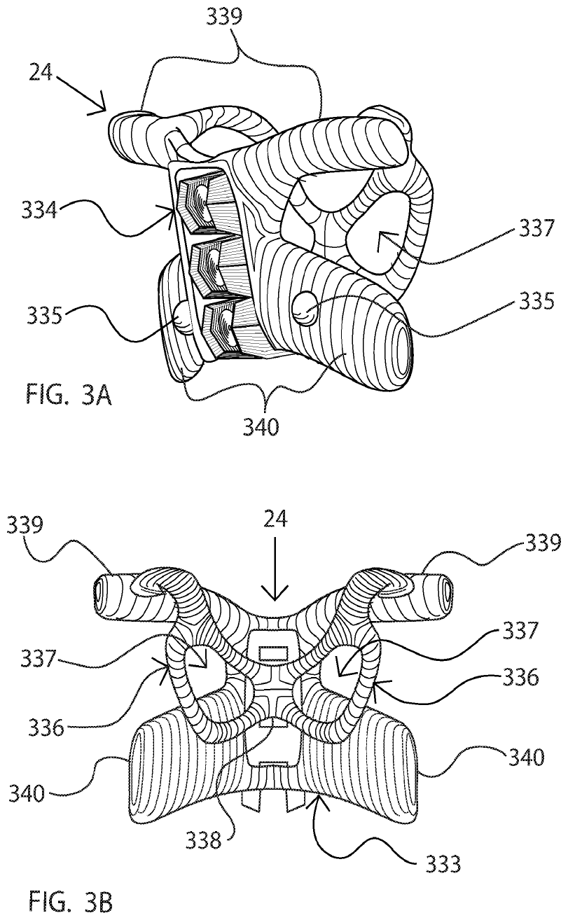

Similarly, the lower oral vestibule seal 264 (or “lower wall”) refers to the wall of the manifold that is configured to reside in the lower anterior oral vestibule of the mouth and creates the air seal in the lower half of the mouth. Its peripheral edge is shaped to provide a notch for the inferior labial frenulum in a central region of the wall. It also includes an attachment configured to physically attach the wall to the lower lip clip, in this case, two studs 266 which pressure fit into the complementary shaped lower lip anchor cut outs 337. Specifically, the inferior labial frenulum notch 265 refers to the notch formed in the lower wall that provides room for the user's inferior labial frenulum. The lower lip clip anchor studs 266 are the mounting mechanisms that provide a way to physically attach to the lower lip clip 24. In this arrangement, the studs comprise two flexible form fitting studs that pressure fit into the complementary shaped lower lip anchor cut outs (337 in FIG. 3A-3B).

Manifold mouth cavity orifice 271 also refers to the central air passageway of the mouthpiece configured to extend between the mouth cavity and the internal cavity 14 of the manifold.

As shown in FIGS. 2A and 2B and 2C, elements of the manifold 60, particularly the body 66 can include a manifold bellow 267, manifold bellow nasal passages 268, a manifold filter orifice 269, a manifold one way valve orifice 270, and a manifold orifice into mouth cavity 271 (i.e., the mouthpiece “air passageway”).

As shown, the body of the manifold comprises a manifold bellow 267, which is a feature of the manifold connected to the nasal base 21. More specifically, manifold bellow 267 can be a portion of the manifold body that is configured to allow for compression and expansion of the manifold in the up/down direction 4 while maintaining an air tight seal from the manifold to the nasal base, whose top terminates into manifold bellows nasal passages 268. As can be appreciated, the bellows 267 can further allow for movement of the nasal base relative to the main portion of the manifold body in the front/back direction 8 and side-to-side direction 6.

The manifold bellow nasal passages 268 are a bifurcated continuation of the air passage defined by the bellows 267 and are shaped to create a pressure-fit seal with the nasal base 21 by insertion into the nasal base airways 524.

Manifold one way valve orifice 270 is an opening in the manifold configured to provide an air tight seal connection to the one way exhalation valve 41. Similarly, the manifold filter orifice 269 is an opening in the manifold configured to provide an air tight seal connection to the filter port 949 of filter 45.

FIG. 1E is a simplified side-view of the internal volume of the breathing apparatus 10, excluding the lip clips and nasal shroud, showing the upper passages of the manifold providing fluidic communication with the person's nasal passage. Also shown is the portion of the manifold 10 provided toward the back end, namely, the mouthpiece 62, which is configured to be received into the mouth and includes a central passageway 271 configured to provide fluidic communication between the person's mouth and the internal volume. Also shown is an outer portion of the manifold that includes one or more openings in the wall of the manifold through which air can be inhaled and/or expired.

A aspect of the disclosed embodiment is the interaction of the mouthpiece/manifold with the armature. The normal base state of the mouthpiece is a resting, slightly open, position as shown in FIG. 2E. Once engaged in the mouth, the shape is responsive to positioning of the lips. If the user closes their mouth, the air passage 271 will also close as shown in FIG. 2F. The manifold responds to the position of the lips and closes orifice 271 completely. If the user opens the mouth, for example by extending the jaw or yawning, the structure of the armature 15 enables the opening of 271 to mirror the movement of the mouth orifice as shown FIG. 2G.

### Lower Lip Clip

An exemplary configuration of the lower lip clip 24 (or “lower clip”) in accordance with one or more embodiments of the invention is shown and described herein in connection with FIGS. 3A-3B, 6B-6C. FIG. 3A depicts a front-left side perspective view of a lower lip clip in accordance with one or more embodiments. FIG. 3B depicts a back-view of the lower lip clip of FIG. 3A. A side view of the lower lip clip is also shown in FIG. 6C.

In an exemplary arrangement, the lower lip clip 24 comprises a chin rest 333, a ball socket gallery 334, a spheroidal filter key 335, a lower anterior oral vestibule anchor 336, a lower anterior oral vestibule anchor cut outs 337, an inferior labial frenulum contour 338, upper arms 339 and a lower base 340.

As shown in FIG. 3B, the chin rest 333 is a structure provided at the bottom end of the lower lip clip, particularly the end that rests against the skin of the face during use. Preferably, the chin rest is configured to rest against the chin, near the mentolabial sulcus, thereby concentrating the weight and energy from the apparatus to the jaw. Accordingly, it can be appreciated that chin rest can extend generally in the lateral direction 6 and generally complement the contour of a chin and have a width suitable for comfortably distributing the forces against the chin. For example, the width of the chin rest can be between 15 to 50 mm, and more preferably 25 to 50 mm.

Again in FIG. 3B, the lower anterior oral vestibule anchor 336 refers to the internal anchor structure of the lower lip clip that rest near the base of the lower anterior oral vestibule. The anchor 336 is provided at an internal end of the Lower Lip Clip arranged to sit between the lower lip and the lower gum. Accordingly, it can be appreciated that lower anterior oral vestibule anchor, particularly its distal end, can be shaped to generally complement the contour of the lip and gums where they intersect and have a width suitable for comfortably distributing the forces against the body. For example, the width of the anchor can be around 10-30 mm, more preferably 15-30 mm Although anchors having larger or smaller widths can be used, however, increasing width (e.g., encompassing the entire oral vestibule) can potentially diminish the ability for clear speech, whereas decreased width can concentrate forces and potentially diminish comfort.

Ball socket gallery 334 is a part of the attachment mechanism configured to connect the expansion device, e.g., expansion bow 23, to the lower lip clip. Preferably, the attachment allows for adjustments, e.g., to provide an adjustable device 10 that can accommodate a broader range of face sizes and adjustable fitment. In this exemplary configuration, it offers three (3) positions. Spheroidal filter key 335 is a protuberance from the lower lip clip that is arranged to key in and lock with a complementary shaped indentation provided in the filter base 948, thereby assisting with the mounting and holding of a filter to the device 10.

Lower anterior oral vestibule anchor cut outs 337 are cut outs formed in the lower lip clip that are shaped match to the mating studs provided on the lower manifolds 266 to create a physical connection between the manifold and the lower lip clip.

Inferior labial frenulum contour 338 is a notch formed in the edge of the lower anterior oral vestibule anchor 336. The lower end of the anchor is contoured to accommodate and provide clearance for the person's inferior labial frenulum. As a result, the lower lip anchor can reside deep in the lower lip anterior oral vestibule fold.

In the upper arms 339, the top functions as a base for the bottom of the breathing manifold and the bottom of the arms frame the top of the mating contour of the filter base. The bottom of the mating contour of the filter base 946 is created by the top of the lower clip base 340. The upper arms 339 as they meet in the center form a contour to allow the shape of the manifold opening 271 in resting form. The connection of the anchor 336 to the upper arms curves around the lip and to the extreme edges of the lower clip arms allowing space for the upper clip 22 to reside in between them when the mouth is closed.

The lower surface 333 of the lower base 340 is the chin rest surface area. The upper surface of the lower base 340 can be configured to receive and support a portion of the filter base, namely, the complementary shaped contour 951 of the filter body 946.

The materials to be used for the construction of the lower lip clip are preferably stable, non-toxic, medical grade materials, e.g. Acrylonitrile Butadiene Styrene (ABS), medical grade plastics, etc. that maintain their molded shape but allow some flexibility.

### Upper Lip Clip

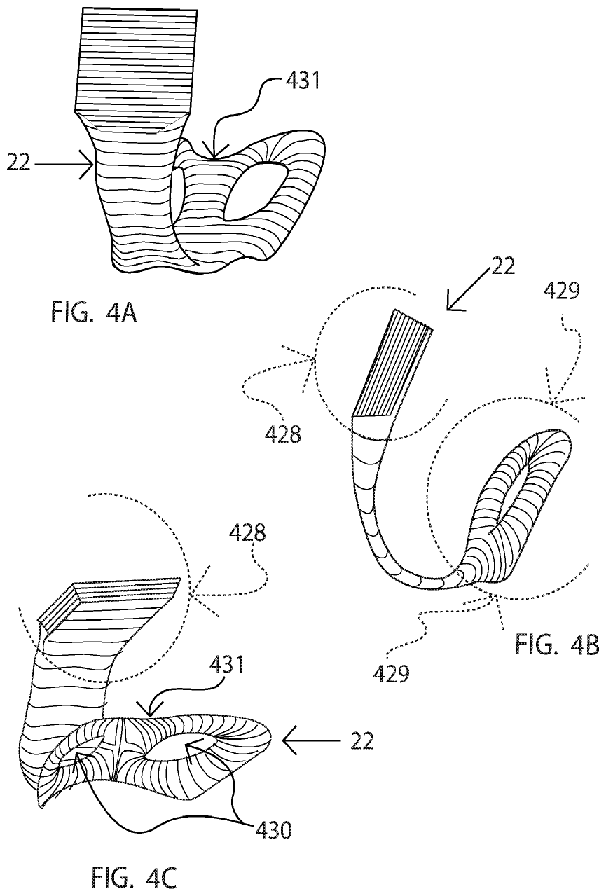

An exemplary configuration of the upper lip clip (or “upper clip”) in accordance with one or more embodiments of the invention is shown and described herein in connection with FIGS. 4A-4C. FIG. 4A depicts a front-left side perspective view of the upper lip clip 22 in accordance with one or more embodiments. FIG. 4B depicts a side-view of the upper lip clip of FIG. 4A. FIG. 4C depicts a perspective view of the upper lip clip of FIG. 4A shown from the top-back view

In an exemplary arrangement, the upper lip clip 22 comprises: dovetail tenon 428, upper anterior oral vestibule anchor 429, upper anterior oral vestibule anchor cut outs 430, superior labial frenulum contour 431.

Dovetail tenon 428 is provided at an external end of the upper lip clip. It is an attachment mechanism configured to attach to the dovetail channel 523 of the nasal base 21. As shown, it is configured to slide thru the channel 523 and have a friction-fit to allow for adjustment of the lip clip relative to the nasal base depending on the dimensions of the individual user's face. Other fixed or adjustable joining mechanisms can be utilized to join the lip clip to the nasal base.

Upper anterior oral vestibule anchor 429 is provided at an internal end of the upper lip clip that sits between the upper lip and the upper gum. Accordingly, it can be appreciated that upper anterior oral vestibule anchor can be shaped to generally complement the contour of the intersection of the gums and lips and have a width suitable for comfortably distributing the forces against the body. For example, the width of the anchor can be around 10 and 30 mm wide and more preferably 15 to 30. Although anchors having larger or smaller widths can be used, however, increasing width (e.g., encompassing the entire oral vestibule) can potentially diminish the ability for clear speech, whereas decreased width can concentrate forces and potentially diminish comfort.

Upper oral vestibule anchor cut outs 430 are perforations that allow a fixed connection to the breathing manifold lip and captures the manifold matching stud 263. Other configurations for attaching the upper lip clip to the manifold lip or “upper wall” are possible.

Superior labial frenulum contour 431 is a notch formed in the edge of the upper anterior oral vestibule anchor 429. The contour is shaped to provide clearance for the person's upper lip labial frenulum. As a result, the upper lip anchor can reside deep in the upper lip anterior oral vestibule fold.

The materials to be used for the construction of the upper lip clip are preferably stable, non-toxic, medical grade materials, e.g. Acrylonitrile Butadiene Styrene (ABS), medical grade plastics, etc. that maintain their molded shape but allow some flexibility.

### Nasal Base:

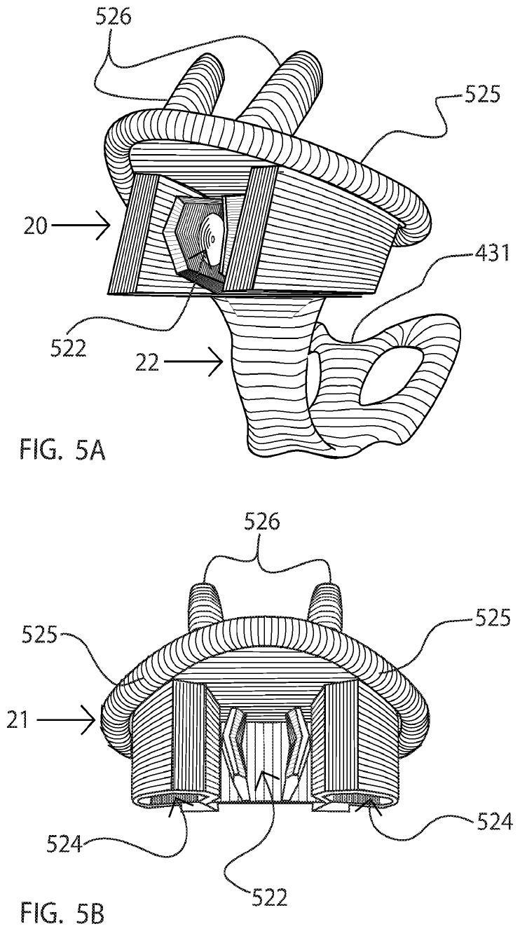

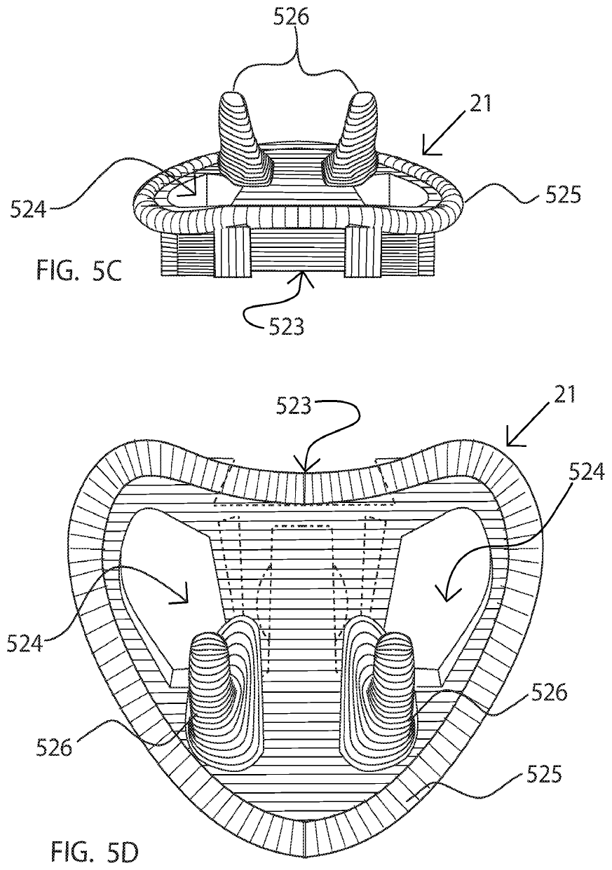

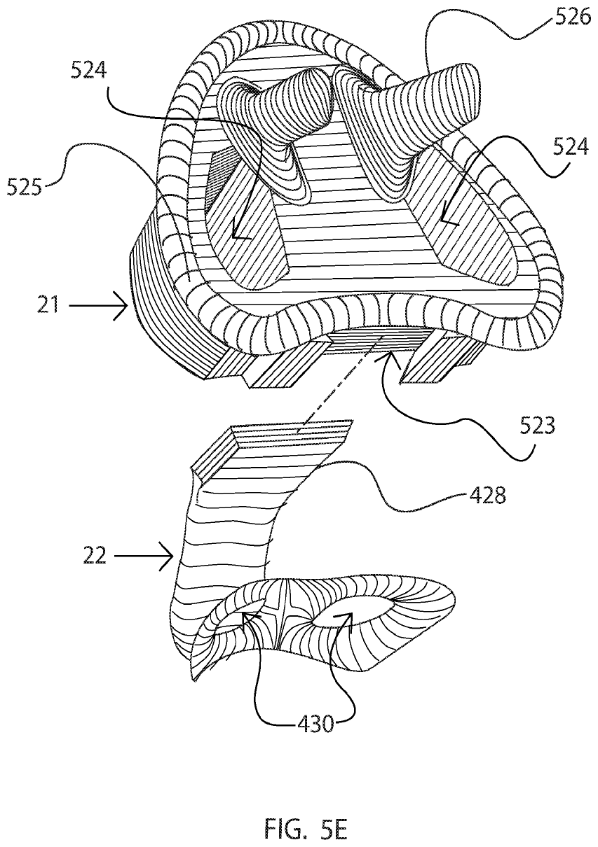

Exemplary components of the nasal base in accordance with one or more embodiments are described herein in connection with FIGS. 5A-5E. FIG. 5A depicts a front-left side perspective view of the nasal base 21 in accordance with one or more embodiments. FIG. 5B depicts a front view of the nasal base of FIG. 5A. FIG. 5C depicts a back view of the nasal base of FIG. 5A. FIG. 5D depicts the nasal base of FIG. 5A from the front-top perspective. FIG. 5E depicts an exploded view of the nasal base of FIG. 5A and the upper lip clip of FIG. 4A shown from the back, left side perspective.

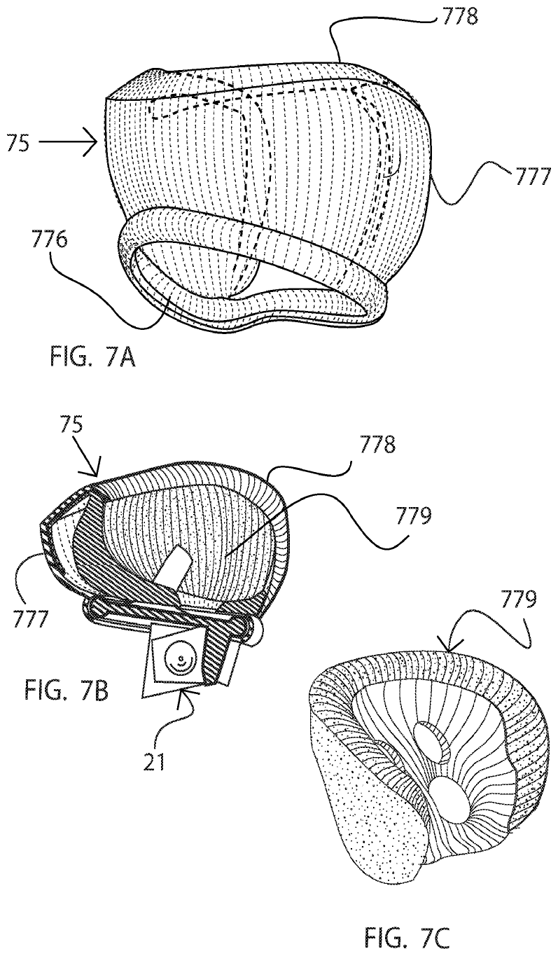

In one arrangement, the nasal base 21 comprises a ball socket 522, dovetail channel 523, nasal airways 524, crown perimeter seal 525, nasal horns 526. The nasal base can also include nasal contour insert (not shown), similar to the contour insert 779 shown in FIG. 7C. The nasal contour insert can be included on the top surface of the nasal base to ensure the seal with the nares and or the nose to the nasal base 21. In one possible embodiment, the insert will be made of an amorphous non-toxic, medical grade plastic, foam or gel. In another possible embodiment the insert can resemble a CPAP nasal pillow.

Ball socket 522, is a socket shaped-structure provided toward a front side of the nasal base and that is configured to receive a ball-shaped end of the expansion bow 522. Although the present configuration implements a ball and socket joint, other suitable mechanical attachments providing freedom of movement in one or more directions between the nasal base and the expansion bow can be used. In addition or alternatively, other flexible materials can be used to connect the components and provide the necessary freedom of movement.

Dove tail channel 523 is a dovetail like channel provided on the back side of the nasal base. The channel 523 is configured to receive the second end of the upper lip clip dovetail tenon 428 allowing to slide in accommodating the difference in length as measured from base of septum to the edge of the lower lip.

Nasal airways 524 provides the structure allowing an airtight connection between the breathing manifold 60 through the nasal base to the nasal passages.

Crown perimeter seal 525 is a structure that extends along the perimeter of the nasal base and configured to provide an airtight connection to the nose shield 75 by connecting with the Nasal base interface 776.

Nasal horns 526 are two semi flexible structures that project upward from the upper surface of the nasal base. The horns 526 are configured to extend through the nares into a respective nostril cavity and help to maintain the nasal base in place. Nasal horns prevent accidental dislodgement (e.g., breaking the seal between the nasal base and the nostrils), say, from the extreme respiratory event such as sneezing, yawning, coughing etc.

In yet another embodiment, the nasal base could be designed to seal the nose from the manifold requiring any gaseous exchange to occur through the mouth orifice. In still another embodiment, all gaseous exchange could be made to occur through the nasal base and the nose but still allowing for speech through the mouth.

The materials to be used for the construction of the Nasal Base are preferably stable, non-toxic, medical grade materials, e.g. Acrylonitrile Butadiene Styrene (ABS), medical grade plastics, etc. that maintain their molded shape but allow some flexibility.

### Expansion Bow

Exemplary components of the expansion bow 23 in accordance with one or more embodiments are described herein in connection with FIG. 6A. FIG. 6A depicts a front-left side perspective view of the expansion bow in both the expanded state (shown on left) and the compressed, loaded or flexed state (shown on right).

In one arrangement, the expansion bow is an elongate member having a semi-rigid material. The bow 23 comprises a ball terminus 632 provided at each end of the bow 23. The bow 23, particularly the bottom ball terminus is configured to be received in the complementary shaped ball socket provided in the ball socket gallery 334 of the lower lip clip. The top ball terminus is similarly received by the complementary shaped ball socket provided on the nasal base, namely, ball socket 522. The ball and socket joining mechanism allows the nasal upper lip module 20, bow 23 and lower lip clip 24 to move freely in multiple directions during use, while remaining connected. Other joints that are suitable for providing relative motion can be used alternatively.

As shown, in use, the bow 23 is bent and joined at its ends to the upper and lower portions of the device. Because the material of the bow has material memory, it naturally forces the upper and lower portions of the device apart, thereby forcing the nasal base against the nose and the lower lip clip against the chin. As can be appreciated the material and the shape of the bow can be defined to provide for a bow having the necessary flexibility to be bent into position and rigidity to exert opposing forces sufficient to sealingly engage the nose and mouth.

The three receiving positions 334 on the lower lip Clip for the expansion bow 23 are designed to allow some customization for size and comfort of the individual wearer. In another embodiment, the relationship between the expansion bow the upper clip and the lower clip could be modified using alternate mechanisms. For example, a ratchet armature, sliding friction armature, etc. In another embodiment, a self-adjusting armature could be designed to allow an automated or semi-automated custom fit by the wearer upon first use for example, by having the wearer introduce the mask and biting down hard to set the adjustment.

Other expansion devices extending between a component of the upper portion of the device (e.g., either the nasal base or upper lip clip) and the lower portion of the device (e.g., the lower lip clip) can also be used.

FIGS. 6B-6D illustrate the assembled armature 15 comprising the nasal base 21, upper lip clip 22, expansion bow 23 and lower lip clip 24 from a perspective front-side view, side view, perspective top-left-back view, respectively.

The exemplary configuration and operation of the armature is further illustrated in FIGS. 1D-1F. FIG. 1D is a cut-away side-view of the armature worn on the face of a user. FIG. 1F illustrates the forces exerted on components of the apparatus 10 and the user's body during use. In particular, FIG. 1F includes a force diagram of the dynamic pivots of the assembly showing the rotational forces that allow the lower clip to pass the weight, stress dynamic onto the chin and allow the upper lip clip to pass forces to the nose and upper lip region. Focusing on FIG. 1F, as noted, the interior anchor portion of the upper and lower lip clips is designed to preferably rest on the base of the respective anterior oral vestibules. The anchor portions resting against the base of respective anterior oral vestibules function as fulcrums 602 and 604 that, under the influence of the expansion bow, cause the clips to rotate thereby achieving their functional roles. The upper lip clip is specifically shaped to rotate in direction 612 about fulcrum 602 creating upward pressure to the nasal base to create a seal on the bottom of the nose. The bottom clip rotating downward along the direction 614 about fulcrum 604 redirecting the weight of the appliance, filters and expansion bow onto the chin and further the masseter muscles. One of the elements that is not readily apparent is that designed relationship avoids applying inordinate stress on the orbicularis oris muscles 99 (i.e., “lip muscles”) allowing them to remain in a relaxed state. This configuration is important for allowing relaxed speech. FIG. 1F further illustrates how the ball and socket joints (between the expansion bow and each of the nasal base and lower lip clip) allow for relative movement of those components, shown by arrows 616 and 618, thereby facilitating free movement of the mouth without disturbing the mouth and nose seal.

As can be appreciated when viewed from the side (FIG. 6C), The lip clips, meaning the lower lip clip 24 and the upper lip clip 22, each form a C-shape. This enables the stresses from the mask to be transferred to the jaw and face rather than impinging on the lip muscles, thereby enabling speech.

### Nasal Shroud

Exemplary components of the nasal shroud and insert in accordance with one or more embodiments are described herein in connection with FIGS. 7A-7C. FIG. 7A is the nasal shroud shown from a front-side perspective. FIG. 7B is a cut-away side view of a cross section of the nasal shroud. FIG. 7C is a top side perspective of the isolated nasal contour insert 779.

In one arrangement, the device can further comprise a nasal shroud 75. The nasal shroud extends from the nasal base and is provided to envelop or seal the person's nose. As shown in FIG. 7A, the shroud 75 includes a nasal base interface 776, a shroud body 777, a shroud upper return 778. The shroud can also include a foam insert 779.

The nasal base interface 776 is a flexible material thickness extending along the contact-point with the crown perimeter seal 525 of the nasal base 21 and configured to create a seal between the nasal shroud 75 and the nasal base. The shroud body 777 is the outer body of the nasal shroud 75 that defines an internal region and that is preferably sufficiently large to encompass the nose of the user. It also is shaped to provide opening in the back to allow the nose to enter the shroud.

In one embodiment the shroud includes a shroud upper return 778, which is the top area of the shroud and made of a material that is flexible enough to conform to the user's nose creating the seal against the outer surface of the nose. In addition or alternatively, as noted previously, the seal can be formed within a trough of the nasal base 21.

Additionally, the shroud can include a compressible nasal contour insert 779, as shown in FIG. 7C in isolation. The insert 779 can be made of an amorphous non-toxic, medical grade plastic, foam or gel.

### One-Way Valve

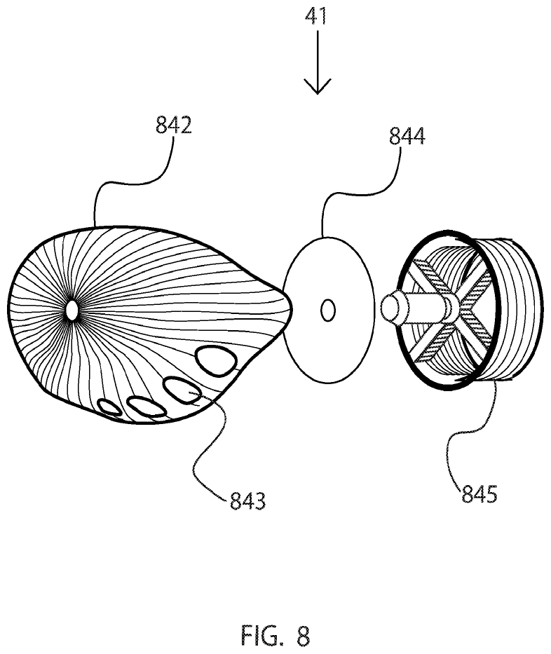

Exemplary components of the exhalation valve in accordance with one or more embodiments are described herein in connection with FIG. 8. FIG. 8 is an exploded diagram of an exemplary valve 41 shown from a front, left side perspective view.

In one arrangement, the valve is a one way exhalation valve 41 that can comprise a number of components including, a one way valve cover 842, a one way valve cover vents 843, a valve membrane 844 and a valve body 845.

The one way exhalation valve 41 can be any number of conventional one-way valves used in breathing apparatuses. Generally, the one way air valves include a channel for air, a supporting non obstructing frame to support the valve membrane, which is a soft membrane that seals the channel by the function of areas of differing air pressure, and a cover to protect the valve membrane.

As shown, one way valve cover 842 is a cover for the valve membrane that allows sufficient air movement while protecting the valve membrane. The one way valve cover vents 843 are perforations on the side over the cover to facilitate air movement. The valve membrane 844 is a membrane of sufficient flexibility to create seal in one direct against the channel frame. The valve body 845 is a rigid base creating a channel for air, attachment to the breathing manifold, and support surfaces for the seal and valve membrane.

### Filter

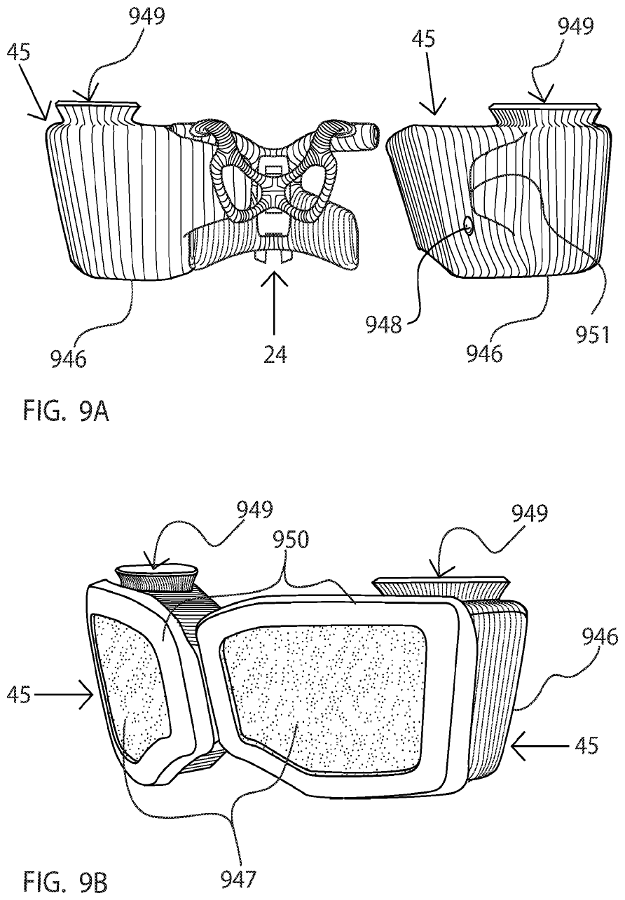

Exemplary components of the filter 45 in accordance with one or more embodiments are described herein in connection with FIGS. 9A-9B. FIG. 9A is a rear-view of two filters 45 and showing the lower lip clip 24 in accordance with one or more embodiments. FIG. 9B is a front left-side perspective view of the two filters of FIG. 9A.

In an exemplary arrangement of a negative pressure respiratory mask or surgical mask, the apparatus 10 is configured to incorporate two filters 45 connected to the manifold 60. Filter 45 comprises a filter body 946, a filter support contour 951, a filter spheroidal key 948, a filter port 949, a filter lid 950, a filter membrane 947. The apparatus 10 is designed in such a way as to filter all incoming air through a filter Unit.

Filter body 946 is a hollow container and represents the structure that holds a filter membrane 947 and creates an airtight seal with the manifold to allow in air thru the filter membrane into the breathing manifold 60. The filter also includes mounting features configured to engage the lower lip clip 24 allowing for a mechanical hold.

Filter support contour 951 is configured to mechanically engage with the complementary shaped structure of the lower lip clip 24. The upper arms 339 and base of the lower bottom 340 lip clip offers sufficient anchoring areas to evolve numerous designs that would allow the transference of forces from the filter to the lower lip clip 24 and subsequent weight bearing by the masseter muscle.

Filter spheroidal key 948 is a spheroidal indentation in the filter body that is configured to be keyed to the lower lip bottom base spheroidal protuberance 335 to hold the filter body in place.

Filter port 949 is an opening in the filter designed to allow for an air seal to be created by attachment to a complementary port provided in the flexible breathing manifold, namely, port 269.

Filter lid 950 is a cover that is configured to fit tight to the filter body 946 capturing the filter Membrane 947 against the filter body and having an opening sufficiently large to allow a comfortable volume of air flow for breathing.

For example and without limitation, the filter membrane 947 can be any commercially available size exclusion membrane conforming to NIOSH classifications for example, N95, R95, O95, etc. In another embodiment, such size exclusion membrane can be further backed by one or more additional chemical or other filters, such as activated carbon and anti-microbial filters, that would impart additional layers of safety depending on the conditions where such a mask is to be used.

In another configuration, the manifold filter orifice 269 could be fitted through a hose or hose-like attachment so that filtration occurs at a remote site for example, on a filtration unit on the individual's chest or through a filtration back pack or even a device on a night stand, etc. This configuration could also enable positive pressure devices. In yet another embodiment useful for emergency applications, the filter 45 can be made to be filled with household materials such as cotton or other textiles easily found around the home to enable functional usage in a variety of situations including emergency situations, e.g. pandemics, where other filter materials are not available.

### Further Configurations and Considerations:

Another unique feature of this invention is shifting the central focus of the supporting structure for the device to the mouth, chin and nose from straps or facial glues. This shift enables at least three major improvements:
1. The number of required sizes for a functional certifiable device can be reduced. This is based on the data gathered that (1) the variation of mouth size/oral vestibule dimensions and (2) the variation of filtrum length between the upper lip and the nose is less amongst individuals than variations seen in overall head and face morphology currently used and needed to secure respiratory devices.
2. By generating the airtight seal of the manifold through the mouth, namely through the interplay of the apparatus with the oral vestibule and the nares/nose, the exemplary configurations improve on (minimize) the level of leakage seen; thereby, increasing the effective filtration of air.
   a. Leakage levels that can be achieved include the following ranges: zero leakage to barely detectable.
   b. Effective filtration that can be achieved can include the following ranges: 95-100 percent of particles down to 0.3 microns or smaller are removed by the filtration media.
3. The Apparatus is designed in such a way that even in the event an individual faints or otherwise losses consciousness the apparatus should remain operational and secured on the face either in negative or positive pressure respirator configurations.

In accordance with one or more embodiments, the apparatus 10 can further include one or more air monitoring sensors provided inside the internal volume 14 of the apparatus 10. These additional sensors can allow continuous monitoring of inspired or expired air for critical external or internal compounds or metabolites or pathogens as well as airflow to determine needed filter changes, to monitor leakage, and even monitor the wearer's health based on exhaled air.

Another configuration of the invention could be for positive pressure applications such as those found in CPAP masks. The removal of head straps would improve comfort to the wearer. The apparatus is constructed in such a way that it should remain on the individuals face and function properly during sleep

Another benefit of the device is that the material used will enable at least the manifold and armature including filter base 45 to be washed, boiled or otherwise cleaned to ensure continued reusability. The filter pads 947 and filter body 45 can be exchangeable to any variety of filters for different uses.

In yet a further embodiment, flavorings can be provided in the manifold to encourage continued use by children or otherwise, e.g. mint for freshness, fruit flavors for children, etc.

In yet a further embodiment, the mouthpiece and size of the apparatus can be adapted for infants and toddlers. For instance, the mouthpiece 62 could incorporate a pacifier outcropping.

Another embodiment can include the insertion of a flexible passageway tubing that could be used by the wearer for drinking from a contained and/or external drinking source

Another embodiment can include features that would allow the internal manifold cavity to be sealed when the mask is taken off or not worn to ensure the air inside the manifold is pristine. The seal could be broken when placed on the users face.

Another embodiment can include incorporation of a face or eye splash shield onto the apparatus.

There are various additional feature possibilities concerning filtration and device coatings/components that can be used in accordance with one or more of the embodiments. A variety of existing and novel filter materials can be used. Materials used to make the breathing tube and armature may also include anti-infective coatings or embedded/blended components/materials that provide anti-infective properties. Examples of current marketed offerings and published techniques for anti-infective agents include: Curad Antiviral Facemask; Silver Shield N95 Facemask; A novel anti-influenza copper oxide containing respiratory face mask; Antiviral metal impregnated activated carbon cloth components; Mask outer face polypropylene non-woven fabric impregnated with bronopol; Antimicrobial nonwoven fabric.

It should be understood that various combination, alternatives and modifications of the present invention could be devised by those skilled in the art. The present invention is intended to embrace all such alternatives, modifications and variances that fall within the scope of the appended claims.

It is to be understood that like numerals in the drawings represent like elements through the several figures, and that not all components and/or steps described and illustrated with reference to the figures are required for all embodiments or arrangements.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

What is claimed is:

1. A breathing apparatus configured to be worn by a person, the mask comprising:
a manifold including:
a body defining an internal chamber,
a sealing mouthpiece extending from the body, the sealing mouthpiece having
an upper wall configured to be received in the upper Oral Vestibule of the mouth,
a lower wall configured to be received in the lower Oral Vestibule of the mouth, wherein the upper and lower walls are configured to sealingly engage an internal surface of the mouth, and
a central passageway located between the upper and lower walls, the central passageway being generally in register with an opening of the mouth, wherein the central passageway provides fluidic communication between the mouth's internal cavity and the internal chamber of the manifold, and
wherein the body is shaped to define one or more ports through which gasses can be exchanged from the internal chamber; and
a nasal base extending from the body and configured to bear against the person's nose, and wherein the nasal base is shaped to define one or more hollow passages providing fluidic communication between the person's nasal passages and the internal chamber of the manifold during use.

2. The breathing apparatus of claim 1, further comprising:
a lower clip configured to be worn on the person's lower lip, the lower clip having a first end arranged for placement within the lower Oral Vestibule, and a second end arranged to bear against an external surface of the person's chin, whereby the lower lip clip distributes a load of the apparatus against the chin, wherein the lower clip is sized and shaped so as to limit interference with the lower lip muscle during use; and

3. The breathing apparatus of claim 2, further comprising:
an upper clip configured to be worn on the person's upper lip, the upper clip having a first end arranged for placement within the upper Oral Vestibule, and a second end external to the person's mouth, wherein the nasal base is mounted to the second end, wherein the upper clip is sized and shaped to locate the nasal base against the underside of the person's nose and to limit interference with the upper lip muscle during use.

4. The apparatus of claim 3, further comprising: an expansion device extending between the upper and lower clips, wherein the expansion device is configured to exert an opposing force between the nasal base and lower clips whereby the expansion device presses the nasal base against the underside of the nose and the second end of the lower clip against the chin.

5. The apparatus of claim 4, wherein the expansion device comprises: a flexible expansion beam coupled to the upper and lower clips using a respective flexible joint, wherein the expansion beam is arranged to exert the opposing force.

6. The apparatus of claim 5, wherein the respective flexible joint is a ball joint.

7. The apparatus of claim 5, wherein the expansion beam is arranged to exert an opposing force that is suitable for holding the apparatus to the person's face without head straps.

8. The apparatus of claim 2, wherein the lower wall of the mouthpiece is joined to the first end of the lower clip, and wherein the upper wall of the mouthpiece is joined to the first end of the upper clip.

9. The apparatus of claim 3, wherein the lower and upper clips are formed of a semi-rigid material.

10. The apparatus of claim 3, wherein the lower and upper lip clips are provided near a mid-point of the mouthpiece in a width-wise direction.

11. The apparatus of claim 1, wherein the central passageway extends axially through the opening of the mouth, and wherein walls defining the central passageway are composed of a flexible material suitable for allowing the central passageway to be opened and collapsed by the person's lips.

12. The apparatus of claim 1, wherein a cross-section of the central passageway generally has the shape of a mouth opening.

13. The apparatus of claim 1, further comprising a nasal shroud mounted to the nasal base, wherein the nasal shroud is configured to sealingly bear against the person's nose.

14. The system of claim 1, wherein the upper wall is sized and shaped such that a peripheral edge of the upper wall is provided within the upper Oral Vestibule toward an intersection of the person's upper lip and gums, and wherein the lower wall is sized and shaped such that a peripheral edge of the lower wall is provided within the lower Oral Vestibule toward an intersection of the person's lower lip and gums.

15. The system of claim 1, wherein the upper and lower walls of the mouthpiece are formed of a material having a thickness and flexibility suitable for providing a seal within the person's mouth and while permitting movement of the lips to facilitate speech.

16. The apparatus of claim 2, wherein the lower clip is shaped to concentrate forces against the chin and near an intersection of the person's lower lip and gums and away from the mouth opening and thereby limiting interference with the lip muscles.

17. The apparatus of claim 3, wherein the upper and lower clips are generally C-shaped.

18. The apparatus of claim 1, wherein the upper and lower walls of the mouthpiece are formed to define a notch near a midpoint.

19. The apparatus of claim 1, wherein the body of the manifold comprises a flexible portion arranged to expand or contract in at least a vertical direction.

20. The apparatus of claim 1, further comprising: a filter attached to a first port and a one-way valve attached to a second port.

21. A device for holding a breathing apparatus to a mouth of a person, the apparatus comprising:
a nasal base configured to bear against an underside of a nose of the person,
an upper clip configured to be worn on an upper lip of the person, the upper clip including:
a first end arranged for placement within an upper Oral Vestibule, and
a second end provided external to the mouth, wherein the nasal base is mounted to the second end, and wherein the upper clip is sized and shaped to locate the nasal base against the underside of the person's nose and limit interference with the person's upper lip muscle during use,
a lower clip configured to be worn on the person's lower lip, the lower clip including:
a first end arranged for placement within the lower Oral Vestibule,
a second end arranged to bear against an external surface of the person's chin and concentrate forces against the chin, and
a lower mount for mounting the lower clip to at least a portion of the breathing apparatus, and wherein the lower clip is sized and shaped to limit interference with the lower lip muscle during use; and
a mount configured to attach the breathing apparatus to one or more of the upper clip, the lower clip and the nasal base.

22. The apparatus of claim 19, further comprising the breathing apparatus, wherein the breathing apparatus comprises a manifold.

23. The apparatus of claim 19, further comprising: an expansion device extending between the nasal base and lower clip, wherein the expansion device is configured to exert an opposing force between the nasal base and lower clip whereby the expansion device presses the nasal base against the underside of the nose and the second end of the lower clip against the chin.
